(19) United States
(12) Patent Application Publication
(10) Pub. No.: US 2019/0262824 A1
(43) Pub. Date: Aug. 29, 2019

(54) Devices and Methods to Reduce Contamination of Fluid Collected Through a Conduit By Absorbent Diversion

(71) Applicant: Juan Nepomuc Walterspiel, Menlo Park, CA (US)

(72) Inventor: Juan Nepomuc Walterspiel, Menlo Park, CA (US)

(21) Appl. No.: 16/282,634

(22) Filed: Feb. 22, 2019

Related U.S. Application Data
(60) Provisional application No. 62/634,819, filed on Feb. 24, 2018.

(51) Int. Cl. B01L 3/00; G01N 33/49; A61B 5/15

(52) U.S. Cl. CPC B01L 3/5023; G01N 33/491; B01L 2400/0403; B01L 2300/0832; A61B 5/150755

(57) ABSTRACT

Fluid sample conduits have absorbing bodies to absorb contaminants in fluid samples or suspensions. The absorbing bodies are shaped to provide surface area for absorption, absorbent materials, and have flow channels for decontaminated portions of the sample to flow through the couduit and be collected. Applications include those in the medical and industrial fields. Absorbing bodies according to this disclosure can also serve the functions of discarding, changing, analyzing, and signaling the composition or physical characteristic or contents of the first, contaminated portion of a fluid sample or suspension through a conduit assembly.

### PRIORITY CLAIM

This United States patent application claims priority to United States Provisional Patent Application No. 62/634,819 filed 24 Feb. 2018 entitled “Devices and Methods to Reduce Contamination of Fluid Collected Through a Conduit by Absorbent Diversion,” Juan Nepomuc Walterspiel, inventor. This application is herewith incorporated fully by reference.

### TECHNICAL FIELD

This disclosure relates to devices and methods for obtaining samples of blood or other biological or non biological fluids and suspensions. More particularly, this disclosure relates to devices and methods for obtaining or transporting or separating or diverting samples of blood or other fluids or suspensions with reduced contamination. Even more particularly, this disclosure relates to devices and methods for obtaining a sample of blood, other fluid, or suspension in which a first portion of the sample is different in nature or composition or is contaminated, and the first, contaminated portion is diverted and sequestrated by an absorbent material, based on the nature of its components, with the remaining portion of a sample or other fluid or suspension, being relatively pure or uncontaminated, then being transported further and collected, and processed.

### BACKGROUND

Analysis and processing of samples of biological fluids is an important aspect of diagnosis and evaluation of many disorders and diseases. Generally, a sample of blood or other body fluid is obtained by inserting a needle or similar device into a blood vessel, infected lesion, suspected malignancy or pathological fluid accumulation and withdrawing a sample into a container to be processed, cultured and/or analyzed by various methods.

A sample of fluid or a suspension that is pumped through a conduit has an initial first or priming portion of the sample that may be or become contaminated and is significantly different or from the rest of the sample. This first portion may have to diverted or eliminated or discarded or changed or the characteristics of its composition sensed or analyzed.

### SUMMARY

I have identified a new problem in the field, namely, that conventional devices and methods for obtaining a sample of blood or other fluid or suspension are inadequate to provide a clean sample. Rather, conventional devices and methods retain a portion of the contaminating skin and its components and with them, microorganisms, their antigens, and their nucleic acids are inadvertently obtained as well and these are usually found in the first portion(s) of the sample.

To solve this problem, I have invented devices and methods where first portion(s) of the sample are absorbed and sequestered and can no longer freely mix with and contaminate the subsequent portions of the sample that will undergo analysis or culture.

These and other aspects and embodiments are described in more detail below, with reference to the attached drawing figures.

### BRIEF DESCRIPTION OF THE DRAWINGS

This disclosure is made with reference to the figures, in which:

FIG. 1A shows a line and partial 3-dimensional drawing of part of an embodiment for a sample collection line with variations of shapes, including spiral, auger-shaped absorbing bodies, and respective linear flow perturbing and deflecting spiral flow channels.

FIG. 1B shows an alternative embodiment showing a conduit having internal planes arranged to direct flow along a spiral path through the conduit.

FIG. 2 shows a line and partial 3-dimensional drawing of an alternative embodiment of part of a sample collection line with a spiral shaped absorbing body and flow deflectors touching a conduit's wall.

FIG. 3 is a line and partial 3-dimensional drawing of an embodiment of a first portion of a sample-collection line or conduit where an absorption body and liner flow deflector is housed in a sample collection conduit that has a spiral form.

FIG. 4 shows a line and partial 3-dimensional drawing of an embodiment of part of a sample collection line with multiple rotated pie shaped absorbing bodies and linear flow deflectors.

FIG. 5 shows a line and partial 3-dimensional drawing of an embodiment of part of a sample line where an absorption body fills the line and has a hollow, spiral flow deflecting conduit within it.

FIG. 6 shows a 3-dimensional drawing of an embodiment having a hollow, twisted flow deflecting conduit within an absorption body.

### DETAILED DESCRIPTION

This disclosure relates generally to a biological specimen line or conduit that contains a contaminant-absorbing, linear flow deflecting body.

### Definitions

The following terms are used as herein indicated unless another section of this disclosure provides a different meaning.

The term “about” refers to an average numerical value ±25%.

The term “absorbent” means a material that absorbs a contaminant.

The term “absorbing body” and “absorbent body” means a device sized to fit within a conduit and having an absorbent material attached thereto.

The term “absorbing sequestering body” means a device within a conduit and having an absorbent attached thereto, so that contaminants are sequestered, and decontaminated fluid is not mixed with contaiminants so sequestered. An absorbing sequestering body may be made of stainless steel, biocompatible polymer and can be coated with an absorbent.

The term “conduit” means a tube which can have attachments at one or two ends to permit the conduit to be attached to another tube. The other tube can be an intravenous line, a drip line from an intravenous feeding bag. Attachments can be Luer type devices. A conduit may be made of stainless steel, biocompatible polymer. A conduit may have an internal coating of an absorbent material.

The term “conduit assembly” means a device comprising a conduit with either an absorbent body within the conduit, or a conduit having an absorbent material attached to the internal surface of the conduit.

The term “sequestering” means the act of removing a contaminant from the fluid, so that contaminants are sequestered, and decontaminated fluid is not mixed with contaiminants so sequestered.

### General Description

In general, the devices and methods of this disclosure include straight, or a tortuous, or winded channel structures within a conduit through which the sample of blood, other fluid, or suspension first passes through a structure containing an absorbent material. A straight or tortuous (twisted) path for the sample changes the the sample's flow dynamics in a way that decreases or prevents the first portion of the sample from being propelled along with the remainder of the sample. Once the first, contaminated portion of the sample is absorbed, the contaminated portion of the sample does not mix with the uncontaminated subsequent portions of the sample. The remaining, uncontaminated portion of the sample will then advance further to be analyzed, or processed, or otherwise used.

It can be desirable for the flow path of a fluid through a conduit assembly to be longer than the straight line distance through the conduit. By providing a longer flow path, the fluid has more opportunities to interact with the absorbent material, and therefore, more contaiminants can be absorbed or sequestered. It can be appreciated that in addition to providing a long flow path, the fluid flow can be turbulent, meaning that there will be increased opportunities for contaminants to make contact with an absorbent material, and be sequestered.

The devices are useful for analysis of cell counts (i.e. in cerebro-spinal fluid or urine), various cultures, nucleic acid sequence determinations, immune stimulation, interferon release assays and many more, whose sensitivity and specificity would be influenced by the presence of material(s) from the skin or in other applications, the tubing within which the fluid or solution to be sampled was contained.

### Aspects of this Disclosure

It is to be understood that any of the following aspects may be implemented singly or in any combination.

One aspect includes a conduit assembly comprising:

a hollow conduit, said conduit containing an absorbing sequestering body retained therein, said absorbing sequestering body comprising an absorbent material adhering to said absorbing sequestering body, said absorbent material to absorb, retain and sequester a contaminated portion of a biological fluid or biological suspension flowing through said conduit, thereby permitting a de-contaminated portion of said fluid or suspension to pass through said conduit and collected for analysis.

Another aspect includes a conduit assembly, said absorbing sequestering body having a spiral shape, configured to interfere with flow of the fluid so that the absorbing body absorbs, sequesters and retains a first contaminated portion of the fluid and so that the remainder of the fluid sample is decontaminated.

An additional aspect includes a conduit assembly, said absorbing sequestering body comprising radiating fins or paddles arranged in staircase spiral form filling the interior of said conduit, thereby defining a spiral shaped flow channel to divert the flow of the fluid so that the absorbing sequestering material absorbs, sequesters and retains a first contaminated portion of the sample.

A further aspect includes a conduit assembly, comprising a hollow conduit having a spiral or twisted shape and having an absorptive material adhered to the interior wall of said conduit, absorptive material to provide a surface for flow of a fluid sample through said conduit so that the absorbent material absorbs, sequesters and retains a first contaminated portion of the fluid sample so that the remainder of the fluid sample is decontaminated and can be collected for analysis.

Another aspect includes a conduit assembly, wherein the absorbing sequestering body comprises a plurality of disks, each having one or more triangular or circular holes through them to permit passage of fluid flow and in contact with said absorbent material.

A still further aspect includes a conduit assembly comprising a hollow conduit, said conduit having a spiral shaped or twisted channel therethrough, said absorbent body configured to deflect flow of the fluid sample so that the absorbing body absorbs, sequesters and retains a first contaminated portion of the fluid so that the remainder of said sample is decontaminated.

An additional aspect includes a conduit assembly, where at least some of the absorbing body contains bound pro-coagulants to facilitate the retention of the first, contaminated, sequestered portion of the fluid being blood.

A still further aspect includes a conduit assembly, wherein at least a portion of said absorbing and sequestering body contains anantibody, enzyme, chemical or isotope, whose interaction with an absorbed or sequestered material produces a detectable change in a characteristic of said absorbed sequestered material.

An additional aspect includes a conduit assembly, wherein said conduit or said absorbing body containing a sensor to detect one or more of the fluid sample's temperature, pressure, content, chemical composition, or pH.

A further aspect includes a conduit assembly, wherein said absorbent material comprises one or more of: cellulose, cellulose linten, cellulose acetate, cellulose nitrate, creped cellulose wadding, blotting paper, filter paper, cross-linked carboxymethylcellulose, comminuted wood fibers, bleached wood pulp, cellulitic wood fibers, spaghmum, diatomaceous earth, absorbent cotton, foamed plastic polymers, low-density polyether, polyethylene, collagen hydroxyapatite, chitosan glutamate, low and high molecular weight sericin and glycine, polyvinyl alcohol, vinyl acetate, vinyl acetate ethylene, superabsorbent polymer(s), sodium polyacrylate, polyacrylamide copolymer, ethylene maleic anhydride copolymer, polyvinyl alcohol copolymers, cross-linked polyethylene oxide and starch grafted copolymers of polyacrylonitrile, polysaccharides, proteins, homo polypeptides (i.e. poly(aspartic acid), poly(glutamic acid), and poly(ε-L-lysine)), pectin, xanthan gum, sodium alginate, calcium alginate, particles of an organic noncellulosic substances selected from the group consisting of blood albumin, egg albumin, starches, algin, karaya, tragacanth and guar gums, natural and synthetic gums of polysaccharide character, chemically modified starches, and hydrocolloidal compositions selected from the group consisting of polyacrylamide, alkali metal salts of hydrolyzed polyacrylamides, and free acid or alkali metal salts of polystyrene sulfonates.

A yet further aspect includes a conduit, where said absorbent body has pores with diameters ranging from about 2 μm to about 25 μm.

An additional aspect includes a method for producing a sample of decontaminated biological fluid, comprising the steps:

a) providing a conduit assembly having a hollow conduit, and optionally an absorbent body therein having an absorbent material attached thereto;

b) initiating flow of a sample of biological fluid through said conduit, a portion of said fluid sample making contact with an absorbent material, said absorbent material either adhered to said conduit or adhered to said absorbing sequestering body;

c) permitting a contaminant to adhere to said absorbent material, so that an uncontaminated portion of said fluid sample flows through the remainder of said conduit; and

d) collecting at least a portion of said uncontaminated fluid.

Further aspects include methods for producing a sample of decontaminated fluid from a biological sample, comprising the steps:

a) providing a conduit assembly of any other aspect of this disclosure;

b) initiating flow of a sample of biological fluid through said conduit, a portion of said fluid sample making contact with an absorbent material, said absorbent material either adhered to said conduit or adhered to said absorbing sequestering body;

c) permitting a contaminant to adhere to said absorbent material, so that an uncontaminated portion of said fluid sample flows through the remainder of said conduit; and

d) collecting at least a portion of said uncontaminated fluid.

Yet further aspects include absorbent material comprising one or more of: cellulose, cellulose lintern, cellulose acetate, cellulose nitrate, creped cellulose wadding, blotting paper, filter paper, cross-linked carboxymethylcellulose, comminuted wood fibers, bleached wood pulp, cellulitic wood fibers, spaghmum, diatomaceous earth, absorbent cotton, foamed plastic polymers, low-density polyether, polyethylene, collagen hydroxyapatite, chitosan glutamate, low and high molecular weight sericin and glycine, polyvinyl alcohol, vinyl acetate, vinyl acetate ethylene, superabsorbent polymer(s), sodium polyacrylate, polyacrylamide copolymer, ethylene maleic anhydride copolymer, polyvinyl alcohol copolymers, cross-linked polyethylene oxide and starch grafted copolymers of polyacrylonitrile, polysaccharides, proteins, homo polypeptides (i.e. poly(aspartic acid), poly(glutamic acid), and poly(ε-L-lysine)), pectin, xanthan gum, sodium alginate, calcium alginate, particles of an organic noncellulosic substances selected from the group consisting of blood albumin, egg albumin, starches, algin, karaya, tragacanth and guar gums, natural and synthetic gums of polysaccharide character, chemically modified starches, and hydrocolloidal compositions selected from the group consisting of polyacrylamide, alkali metal salts of hydrolyzed polyacrylamides and free acid or alkali metal salts of polystyrene sulfonates.

### DESCRIPTION OF SPECIFIC EMBODIMENTS

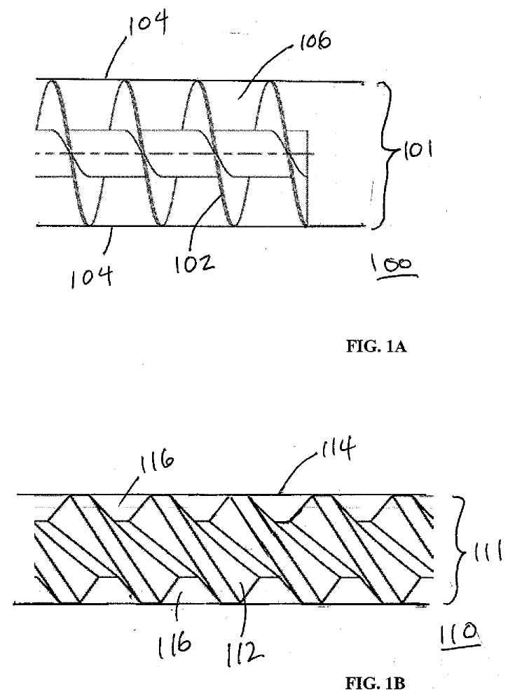

FIG. 1A depicts a line and partial 3-dimensional drawing 100 of an embodiment of sample-collection line or conduit. Drawing 100 depicts conduit 101 having walls 104. Conduit 101 is filled with the spiral, auger shaped absorption body 102. Absorption body 102 is configured within the conduit to produce a spiral path for a biological sample to be passed through conduit 101. Upon entering a first end of conduit 101, the first part of a biological sample is separated by absorption by absorbent body 102, with the remainder of the biological sample passing through a spiral path to the other end of conduit 101 and then out of conduit 101 and into a sample collection vessel (not shown).

FIG. 1B depicts an alternative embodiment 110 having conduit 111 with sidewalls 114, and absorption bodies of planes of material attached together to produce a spiral path 116 through conduit 111. As with embodiment 100 of FIG. 1A, upon entering a first end of conduit 111, the first part of a biological sample is separated by absorption by absorbent body 112, with the remainder of the biological sample passing through a spiral path to the other end of conduit 111, and then out of conduit 111 into a sample collection vessel (not shown).

FIG. 2 depicts a line and partial 3-dimensional drawing of an embodiment 200 of a sample-collection line or conduit 201, a first portion of sample absorption body and linear flow deflector 202 with multiple paddles 208 that are arranged in a spiral configuration. Paddles 208 touch the inner wall of a sample-collection line or conduit 204. As with embodiment 100 of FIG. 1A and FIG. 1B, upon entering a first end of conduit 201, the first part of a biological sample is separated by absorption by paddles 208 of linear flow deflector 202, with the remainder of the biological sample passing through the interior of conduit 211 and exiting conduit 211 and into a collection vessel (not shown).

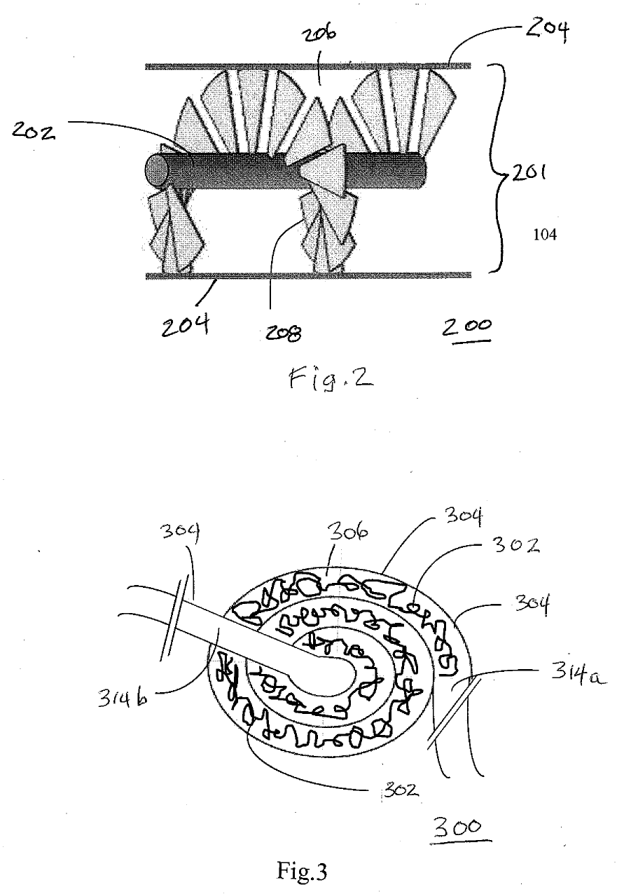

FIG. 3 depicts a line and partial 3-dimensional drawing of an embodiment 300 of a sample-collection line having inflow end 314a of a conduit and outflow end 314b. The remainder of the conduit is shown in a helical or spiral configuration, with the conduit having walls 304. Within walls 304, absorption body 302 is shown having rope-like or undulating shape that does not completely fill the conduit between walls 304. Surrounding absorption body 302, space 306 is shown. Upon entering inflow end 314a, a biological sample comes into contact with absorption body 302, and the first part of the biological sample is absorbed or sequestered within absorption body 302. The remainder of the biological sample passes through space 306 of the conduit and through outflow end 314b and into a collection vessel (not shown).

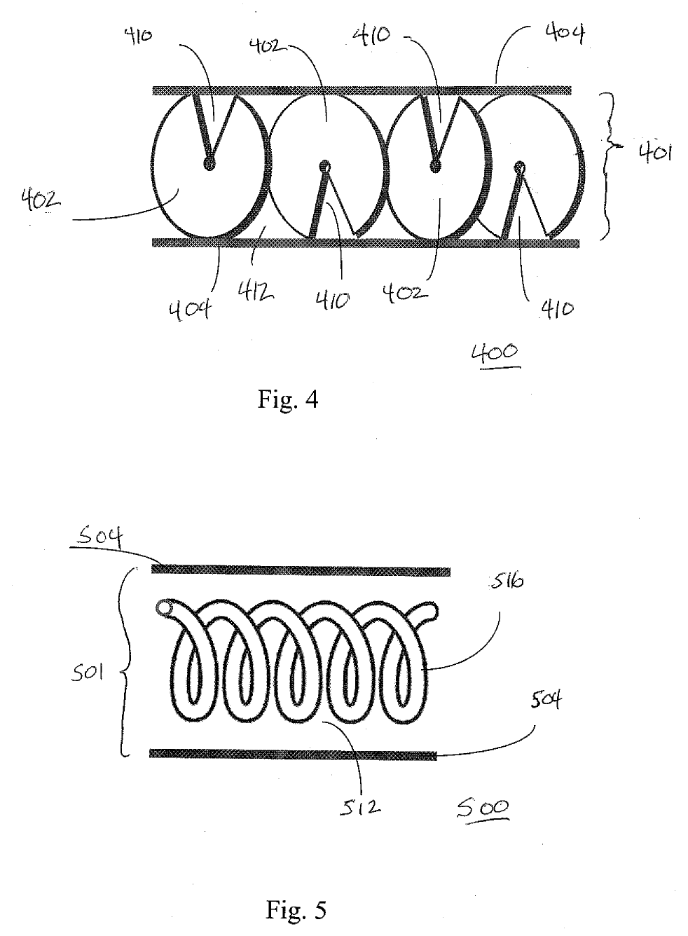

FIG. 4 depicts a line and partial 3-dimensional drawing of an embodiment 400 of a sample-collection line or conduit 401 having walls 404 that have within them, disc-shaped absorption bodies 402, that have slits 410. Absorption bodies 402 are arrayed with respect to each other, so that the slits 410 are not axially superimposed over each other. Conduit 401 contains space 412 that is continuous in the interior of conduit 401. Upon entering a first end of conduit 401, the first portion of a biological sample makes contact with absorption bodies 402, where a contaminated portion of the biological sample is absorbed or sequestered. The remainder of the biological passes through slits 410, and through space 412 of conduit 401, out of the other end of conduit 401 and into a sample collection vessel (not shown).

FIG. 5 depicts a line and partial 3-dimensional drawing of embodiment 500, with sample collection conduit 501 having walls 504 defining space 512. Space 512 has within it a spiral shaped absorbent body 516 coated with an absorbent material. Absorbent body 516 impedes the linear flow of a sample fluid and contaminants are absorbed and sequestered by the absorbent material. Upon entering a first end of conduit 501, the first part of a biological sample makes contact with absorption body 516, where contaminants are absorbed and sequestered. The remainder of the biological fluid moves through space between walls 504, through the other end of conduit 501 and into a sample collection vessel (not shown).

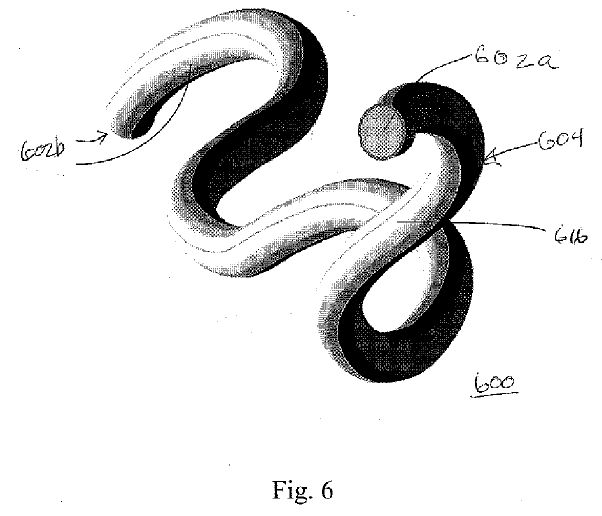

FIG. 6 depicts a 3-dimensional drawing of embodiment 600 of sample conduit 616 having a twisted internal path, within absorption material attached to the interior surface of conduit 616 therein (not shown). Upon entering into a first end 602a of conduit 616 the first part of a biological sample makes contact with the absorbent material within conduit 616, where the first part of the biological sample is absorbed and sequestered. The remainder of the biological sample exits through outflow end 602b and into a sample collection vessel (not shown).

### Use

To use the conduit assemblies of this disclosure, a fluid or suspension is collected from a source and is introduced into one end of a conduit. Upon introduction of the sample into the conduit, a first portion of the sample makes contact with an absorbent body or absorbent material within the conduit. By slowing the flow of the sample, absorption of a first portion of the sample with its contaminants is facilitated. After making contact with the absorbent material, suspended contaminants are absorbed into or sequestered within the absorbent body, and the remainder of the sample, now being decontaminated, flows through the remainder of the conduit, and can then be collected for analysis.

### Materials

The material compositions of the absorbing bodies are generally chosen based on the absorptive characteristics, their swelling or non-swelling characteristics, hydrophobic or hydrophilic surfaces, clotting initiation properties and their abilities to be manufactured into desired shapes. Some absorptive materials can have pores to increase the surface area to volume ratio, thereby promoting more efficient absorption. Absorbing material mixtures or layers thereof and the respective dimensions can be chosen depending upon the desired sample(s) to be de-contaminated, the shapes of the conduits, and the non-interference of the absorbent material with the analytes, e.g., specific proteins, lipids, electrolytes, and any organisms or DNA or RNA sequence of interest in the components present in de-contaminated samples. Such organisms of interest are those present in the bloodstream, which may cause fever and/or sepsis, for which identification is of importance to develop organism-specific responses in a patient.

Materials for absorption bodies may be single compositions or combinations, and may be in single or multiple layers. In some embodiments, absorbent materials can have pore diameters ranging from about 2 μm to about 25 μm to sequester particulates, including red and white blood cells, as well as platelets in a sample.

Exemplary absorptive materials include cellulose, cellulose lintens, creped cellulose wadding, cellulose acetate, cellulose nitrate, blotting paper, filter paper, cross-linked carboxymethylcellulose, comminuted wood fibers, bleached wood pulp, cellulitic wood fibers, spaghmum, diatomaceous earth, absorbent cotton, foamed plastic polymers, low-density polyether, polyethylene, collagen hydroxyapatite, chitosan glutamate, low and high molecular weight sericin and glycine, polyvinyl alcohol, vinyl acetate, vinyl acetate ethylene, superabsorbent polymer(s), sodium polyacrylate, polyacrylamide copolymer, ethylene maleic anhydride copolymer, polyvinyl alcohol copolymers, cross-linked polyethylene oxide and starch grafted copolymers of polyacrylonitrile, polysaccharides, proteins, homo polypeptides (i.e. poly(aspartic acid), poly(glutamic acid), and poly(ε-L-lysine)), pectin, xanthan gum, sodium alginate, calcium alginate, particles of an organic noncellulosic substances selected from the group consisting of blood albumin, egg albumin, starches, algin, karaya, tragacanth and guar gums, natural and synthetic gums of polysaccharide character, chemically modified starches, and hydrocolloidal compositions selected from the group consisting of polyacrylamide, alkali metal salts of hydrolyzed polyacrylamides and free acid or alkali metal salts of polystyrene sulfonates.

### Applications

The foregoing descriptions have broad applications. For example, while examples disclosed herein disclose the absorption and sequestration of first portions of biological samples, this can also be used to clean or alter or signal the chemical composition or property of the initial portion of any fluid or solution that is for the first time pumped or transported through a conduit.

An absorbing body can also function in reverse, in embodiments that are loaded with useful chemical, enzymes or antibodies and there like that change and signal the composition and character of the fluids or suspension that are pumped through them at various speeds and directions. They can also be employed to dampen sudden changes in their composition that which would make them more useful for applications that an ordinary person who is skilled in the art would recognize.

Accordingly, the descriptions herein are meant only to be exemplary and are not intended to suggest that the scope of the disclosure, including the claims, is limited to these embodiments.

### INDUSTRIAL APPLICABILITY

Devices and methods of this disclosure for sequestering the first, contaminated portion of a sample is useful in the medical field, including analysis of components of the sample, which is valuable for making diagnosis and/or evaluating therapies.

What is claimed is:

1. A conduit assembly comprising:
a hollow conduit, said conduit containing an absorbing sequestering body retained therein, said absorbing sequestering body comprising an absorbent material adhering to said absorbing sequestering body, said absorbent material to absorb, retain and sequester a contaminated portion of a biological fluid or biological suspension flowing through said conduit, thereby permitting a de-contaminated portion of said fluid or suspension to pass through said conduit and collected for analysis.

2. The conduit assembly of claim 1, said absorbing sequestering body having a spiral shape, configured to interfere with flow of the fluid so that the absorbing body absorbs, sequesters and retains a first contaminated portion of the fluid and so that the remainder of the fluid sample is decontaminated.

3. The conduit assembly of claim 1 said absorbing sequestering body comprising radiating fins or paddles arranged in staircase spiral form filling the interior of said conduit, thereby defining a spiral shaped flow channel to divert the flow of the fluid so that the absorbing sequestering material absorbs, sequesters and retains a first contaminated portion of the sample.

4. A conduit assembly, comprising a hollow conduit having a spiral or twisted shape and having an absorptive material adhered to the interior wall of said conduit, absorptive material to provide a surface for flow of a fluid sample through said conduit so that the absorbent material absorbs, sequesters and retains a first contaminated portion of the fluid sample so that the remainder of the fluid sample is decontaminated and can be collected for analysis.

5. The conduit assembly of claim 1, wherein the absorbing sequestering body comprises a plurality of disks, each having one or more triangular or circular holes through them to permit passage of fluid flow and in contact with said absorbent material.

6. The conduit assembly of claim 1, said conduit having a spiral shaped or twisted channel therethrough, said absorbent body configured to deflect flow of the fluid sample so that the absorbing body absorbs, sequesters and retains a first contaminated portion of the fluid so that the remainder of said sample is decontaminated.

7. The conduit assembly of claim 1, where at least some of the absorbing body contains bound pro-coagulants to facilitate the retention of the first, contaminated, sequestered portion of the fluid being blood.

8. The conduit assembly of claim 1, wherein at least a portion of said absorbing and sequestering body contains an antibody, enzyme, chemical or isotope, whose interaction with an absorbed or sequestered material produces a detectable change in a characteristic of said absorbed sequestered material.

9. The conduit assembly of claim 1, wherein said conduit or said absorbing body containing a sensor to detect one or more of the fluid sample's temperature, pressure, content, chemical composition, or pH.

10. The conduit assembly of claim 1, wherein said absorbent material comprises one or more of: cellulose, cellulose linten, cellulose acetate, cellulose nitrate, creped cellulose wadding, blotting paper, filter paper, cross-linked carboxymethylcellulose, comminuted wood fibers, bleached wood pulp, cellulitic wood fibers, spaghmum, diatomaceous earth, absorbent cotton, foamed plastic polymers, low-density polyether, polyethylene, collagen hydroxyapatite, chitosan glutamate, low and high molecular weight sericin and glycine, polyvinyl alcohol, vinyl acetate, vinyl acetate ethylene, superabsorbent polymer(s), sodium polyacrylate, polyacrylamide copolymer, ethylene maleic anhydride copolymer, polyvinyl alcohol copolymers, cross-linked polyethylene oxide and starch grafted copolymers of polyacrylonitrile, polysaccharides, proteins, homo polypeptides (i.e. poly(aspartic acid), poly(glutamic acid), and poly(ε-L-lysine)), pectin, xanthan gum, sodium alginate, calcium alginate, particles of an organic noncellulosic substances selected from the group consisting of blood albumin, egg albumin, starches, algin, karaya, tragacanth and guar gums, natural and synthetic gums of polysaccharide character, chemically modified starches, and hydrocolloidal compositions selected from the group consisting of polyacrylamide, alkali metal salts of hydrolyzed polyacrylamides and free acid or alkali metal salts of polystyrene sulfonates.

11. The conduit of claim 1, where said absorbent body has pores with diameters ranging from about 2 μm to about 25 μm.

12. A conduit for obtaining a sample of decontaminated biological fluid, comprising:
a) a hollow conduit; having an interior surface and an exterior surface; and
b) an absorbent material attached to the interior surface of said conduit, said absorbent material sized to not occlude the conduit and therefore to permit fluid flow through said conduit.

13. A method for producing a sample of decontaminated fluid from a biological sample, comprising the steps:
a) providing a conduit assembly of claim 1;
b) initiating flow of a sample of biological fluid through said conduit, a portion of said fluid sample making contact with an absorbent material, said absorbent material either adhered to said conduit or adhered to said absorbing sequestering body;
c) permitting a contaminant to adhere to said absorbent material, so that an uncontaminated portion of said fluid sample flows through the remainder of said conduit; and
d) collecting at least a portion of said uncontaminated fluid.

14. The method of claim 13, wherein said absorbent material comprises one or more of: cellulose, cellulose lintern, cellulose acetate, cellulose nitrate, creped cellulose wadding, blotting paper, filter paper, cross-linked carboxymethylcellulose, comminuted wood fibers, bleached wood pulp, cellulitic wood fibers, spaghmum, diatomaceous earth, absorbent cotton, foamed plastic polymers, low-density polyether, polyethylene, collagen hydroxyapatite, chitosan glutamate, low and high molecular weight sericin and glycine, polyvinyl alcohol, vinyl acetate, vinyl acetate ethylene, superabsorbent polymer(s), sodium polyacrylate, polyacrylamide copolymer, ethylene maleic anhydride copolymer, polyvinyl alcohol copolymers, cross-linked polyethylene oxide and starch grafted copolymers of polyacrylonitrile, polysaccharides, proteins, homo polypeptides (i.e. poly(aspartic acid), poly(glutamic acid), and poly(ε-L-lysine)), pectin, xanthan gum, sodium alginate, calcium alginate, particles of an organic noncellulosic substances selected from the group consisting of blood albumin, egg albumin, starches, algin, karaya, tragacanth and guar gums, natural and synthetic gums of polysaccharide character, chemically modified starches, and hydrocolloidal compositions selected from the group consisting of polyacrylamide, alkali metal salts of hydrolyzed polyacrylamides and free acid or alkali metal salts of polystyrene sulfonates.
